(19) United States
(12) Patent Application Publication
(10) Pub. No.: US 2023/0028240 A1
(43) Pub. Date: Jan. 26, 2023

(54) AI-BASED CLOUD PLATFORM SYSTEM FOR DIAGNOSING MEDICAL IMAGE

(71) Applicant: DEEPNOID CO., LTD., Seoul (KR)

(72) Inventors: Woo-Sik CHOI, Gimpo-si (KR); Tae-Gyu KIM, Yongin-si (KR); Won-Woo JUNG, Seoul (KR); Seong-Woo SEO, Seoul (KR); Ji-Young OH, Bucheon-si (KR)

(21) Appl. No.: 17/293,186

(22) PCT Filed: Mar. 22, 2021
(86) PCT No.: PCT/KR2021/003479

§ 371 Date: Sep. 29, 2021

(30) Foreign Application Priority Data
Apr. 16, 2020 (KR) 10-2020-0045740

(51) Int. Cl. G16H 30/40; G06T 7/10; G06T 7/00; G06V 10/764; G06F 3/0486; G06F 3/04817

(52) U.S. Cl. CPC G16H 30/40; G06T 7/10; G06T 7/0012; G06V 10/764; G06F 3/0486; G06F 3/04817; G06T 2207/20081

(57) ABSTRACT

Provided is a cloud platform system for reading a medical image, the cloud platform system including: multiple image processing modules pre-programmed to perform preprocessing of the medical image and modularized; multiple artificial intelligence modules in which an artificial intelligence algorithm is pre-programmed and modularized; multiple layer modules in which layers applied to a configuration of the artificial intelligence algorithm are modularized by function; a learning model design unit providing a graphical user interface for designing an artificial intelligence-based learning model to a user terminal that has had access through a web browser; and a reading model generation unit generating a reading model by training the learning model designed by the learning model design unit.

### TECHNICAL FIELD

The present disclosure relates to an artificial intelligence-based cloud platform system for reading a medical image. More particularly, the present disclosure relates to an artificial intelligence-based cloud platform system for reading a medical image, wherein multiple users access the system and the system supports design and generation of an artificial intelligence-based reading model.

### BACKGROUND ART

Various algorithms using an artificial intelligence technique have been developed for a long time. In particular, various techniques for processing big data by applying a deep learning algorithm have been developed recently, and success cases of applying such techniques have been increasing gradually.

So far, there have been active attempts to receive help in making clinical decisions by applying artificial intelligence to reading medical images. In particular, there have been developed methods of helping clinicians to make decisions by applying artificial intelligence algorithms to reading medical images acquired from diagnosis devices using X-rays, ultrasonography, computed tomography (CT), magnetic resonance imaging (MRI), positron emission tomography (PET), and magnetic resonance angiography (MRA).

It is known that an auxiliary diagnosis system for classifying, through artificial intelligence, whether tissue shown in a medical image is normal or abnormal, or whether a tumor is malignant or benign has an increased rate of lesion detection, compared to the case in which only a radiologist reads a medical image. For such classification, naive Bayes, support-vector machines (SVMs), artificial neural networks (ANNs), and hidden Markov models (HMMs) are mainly used, which are algorithms that automatically classify the presence or absence of lesions.

Machine learning algorithms may be used as artificial intelligence algorithms, and machine learning may be roughly divided into supervised learning and unsupervised learning. Such machine learning algorithms may be used to generate a reading model (or a prediction model), and the generated reading model may be used to construct a system for inferring whether normality or abnormality for a medical image takes place. Recently, research for generating a reading model enabling a more accurate diagnosis has been conducted.

As described above, the artificial intelligence algorithms are divided into supervised learning and unsupervised learning. Examples of supervised learning include classification, a decision tree, a k-nearest neighbors algorithm (k-NN), a neural network, and support-vector machines (SVMs). Examples of unsupervised learning include clustering. In addition, semi-supervised learning and reinforcement learning are also known as artificial intelligence algorithms.

As the artificial intelligence algorithms used for reading medical images, for example, a diagnosis of a lesion or disease, the following algorithms are mainly used: a classification algorithm, an object detection algorithm, and a segmentation algorithm. The classification algorithm has been developed in various ways, such as ResNet, DenseNet, and MobileNet.

Due to the presence of such a wide variety of algorithms, it is difficult to design a learning model for reading medical images unless the user is an expert in the field of artificial intelligence, for example, an expert who can code an algorithm manually and design a learning model.

In addition, medical images are personal information, so it is difficult to acquire medical images, and it is also difficult to acquire enough medical images to increase the reading accuracy of a learning model.

### DISCLOSURE

### Technical Problem

Accordingly, the present disclosure has been made keeping in mind the above problems occurring in the related art, and the present disclosure is directed to providing an artificial intelligence-based cloud platform system for reading a medical image, the system being capable of providing various types of medical images, providing modularized image processing algorithms for preprocessing or postprocessing and modularized artificial intelligence algorithms, and providing an environment in which the user is able to easily design an artificial intelligence-based learning model.

### Technical Solution

According to the present disclosure, there is provided a cloud platform system for reading a medical image, the cloud platform system including: multiple image processing modules pre-programmed to perform preprocessing of the medical image and modularized; multiple artificial intelligence modules in which an artificial intelligence algorithm is pre-programmed and modularized; multiple layer modules in which layers applied to a configuration of the artificial intelligence algorithm are modularized by function; a learning model design unit providing a graphical user interface for designing an artificial intelligence-based learning model to a user terminal that has had access through a web browser; and a reading model generation unit generating a reading model by training the learning model designed by the learning model design unit, wherein the learning model design unit configured to: display a layer list display window in which a list of the multiple layer modules is displayed and an artificial intelligence design window in the graphical user interface; display, when the list displayed in the layer list display window is dragged and dropped into the artificial intelligence design window, layer icons of the layer modules in the artificial intelligence design window; and generate the artificial intelligence module using line connection between the layer icons as a data flow when the layer icons displayed in the artificial intelligence design window are connected in a line.

Herein, the cloud platform system may further include: multiple layer blocks composed of at least two or more layers and modularized for generating the artificial intelligence module, wherein the learning model design unit may be configured to: display a list of the multiple layer blocks in the layer list display window; and support generation of the layer block through drag and drop of the list of the layer modules displayed in the layer list display window into the artificial intelligence design window, and through line connection.

In addition, the learning model design unit may be configured to: display a module list display window in which a list of the multiple image processing modules and a list of the multiple artificial intelligence modules are displayed and a learning model design window in the graphical user interface; display, when at least one of the lists displayed in the module list display window is dragged and dropped into the learning model design window, module icons corresponding to the image processing modules or the artificial intelligence modules in the learning model design window; and generate the learning model using line connection between the module icons as a data flow when the module icons are connected in a line.

In addition, the learning model design unit may be configured to: display a view button when the module icon corresponding to the artificial intelligence module is selected; and display, when the view button is selected, the layer list display window and the artificial intelligence design window in the graphical user interface, displaying line connection to the layer modules and/or the layer blocks constituting the artificial intelligence module in the artificial intelligence design window.

In addition, the cloud platform system may further include: a dataset storage unit in which multiple datasets classified according to at least one among a body part, a type of modality, a type of disease to be read, and a type of image dimension are stored, wherein the learning model design unit may be configured to: display a list of the multiple datasets in the module list display window; display, when the dataset displayed in the module list display window is dragged and dropped into the learning model design window, a data icon in the artificial intelligence design window in response to drag and drop; and generate, when at least one of the module icons is connected to the data icon in a line, the learning model by using line connection to the module icon as a data flow, and the reading model generation unit may be configured to generate the reading model by training the learning model generated by the learning model design unit, with the dataset corresponding to the data icon.

In addition, the list of the multiple image processing modules displayed in the module list display window may be displayed, being divided into multiple preprocessing function-based groups, and the preprocessing function-based groups may include at least two of the following: a color preprocessing group in which a list of the image processing modules operating for an 8-bit or more color image is displayed, a grayscale preprocessing group in which a list of the image processing modules operating for an 8-bit black-and-white image is displayed, and a binary preprocessing group in which a list of the image processing modules operating for a 1-bit black-and-white image is displayed.

In addition, the multiple artificial intelligence modules displayed in the module list display window may be displayed, being divided into multiple artificial intelligence function-based groups, and the artificial intelligence function-based groups may include: a 2D classification group in which a list of the artificial intelligence modules performing classification of a 2D image is displayed, a 3D classification group in which a list of the artificial intelligence modules performing classification of a 3D image is displayed, a 2D detection group in which a list of the artificial intelligence modules performing object detection of a 2D image is displayed, a 3D detection group in which a list of the artificial intelligence modules performing object detection of a 3D image is displayed, a 2D segmentation group in which a list of the artificial intelligence modules performing segmentation of a 2D image is displayed, and a 3D segmentation group in which a list of the artificial intelligence modules performing segmentation of a 3D image is displayed.

### Advantageous Effects

According to the present disclosure with the above configuration, provided is an artificial intelligence-based cloud platform system for reading a medical image, the system being capable of providing various types of medical images, providing modularized image processing algorithms for preprocessing or postprocessing and modularized artificial intelligence algorithms, and providing an environment in which the user is able to easily design an artificial intelligence-based learning model.

### DESCRIPTION OF DRAWINGS

FIGS. 1 and 2 are diagrams illustrating a configuration of an artificial intelligence-based cloud platform system according to an embodiment of the present disclosure, and

FIGS. 3 to 24 are diagrams illustrating a graphical user interface that an artificial intelligence-based cloud platform system according to an embodiment of the present disclosure provides.

### BEST MODE

The present disclosure relates to a cloud platform system for reading a medical image, the cloud platform system including: multiple image processing modules pre-programmed to perform preprocessing of the medical image and modularized; multiple artificial intelligence modules in which an artificial intelligence algorithm is pre-programmed and modularized; multiple layer modules in which layers applied to a configuration of the artificial intelligence algorithm are modularized by function; a learning model design unit providing a graphical user interface for designing an artificial intelligence-based learning model to a user terminal that has had access through a web browser; and a reading model generation unit generating a reading model by training the learning model designed by the learning model design unit, wherein the learning model design unit configured to display a layer list display window in which a list of the multiple layer modules is displayed and an artificial intelligence design window in the graphical user interface; display, when the list displayed in the layer list display window is dragged and dropped into the artificial intelligence design window, layer icons of the layer modules in the artificial intelligence design window; and generate the artificial intelligence module using line connection between the layer icons as a data flow when the layer icons displayed in the artificial intelligence design window are connected in a line.

### MODE FOR INVENTION

The present disclosure may be modified in various ways and implemented by various embodiments, and specific embodiments are shown in the drawings and will be described in detail.

However, the present disclosure is not limited thereto, and the exemplary embodiments can be construed as including all modifications, equivalents, or substitutes in a technical concept and a technical scope of the present disclosure.

The terms used in the present application are merely used to describe particular embodiments, and are not intended to limit the present disclosure. An expression used in the singular encompasses the expression of the plural, unless it has a clearly different meaning in the context. In the present application, it is to be understood that terms such as “including”, “having”, etc. are intended to indicate the existence of the features, numbers, steps, operations, elements, parts, or combinations thereof disclosed in the specification, and are not intended to preclude the possibility that one or more other features, numbers, steps, operations, elements, parts, or combinations thereof may exist or may be added.

Unless otherwise defined, all terms including technical and scientific terms used herein have the same meaning as commonly understood by one of ordinary skill in the art to which the present disclosure belongs. It will be further understood that terms used herein should be interpreted as having a meaning that is consistent with their meaning in the context of the relevant art and will not be interpreted in an idealized or overly formal sense unless expressly so defined herein.

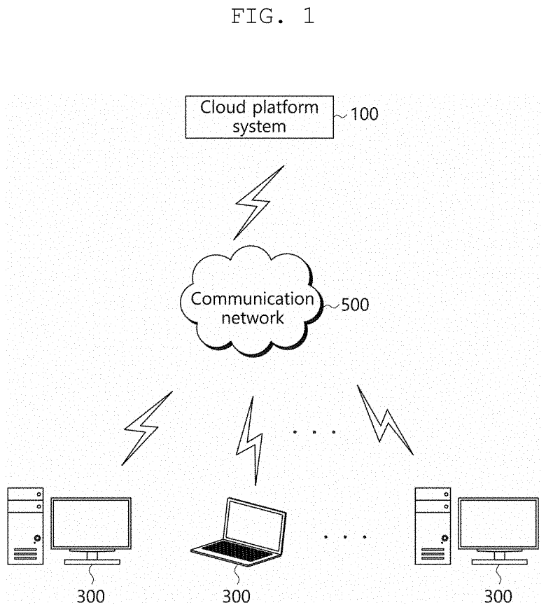

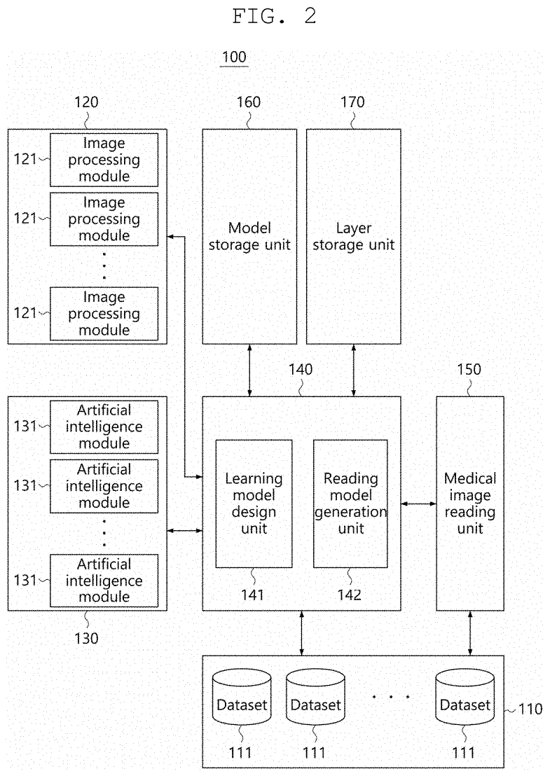

FIG. 1 is a diagram illustrating a use structure of an artificial intelligence-based cloud platform system 100 according to an embodiment of the present disclosure. FIG. 2 is a diagram illustrating an example of a configuration of an artificial intelligence-based cloud platform system 100 according to an embodiment of the present disclosure.

Referring to FIG. 1, multiple user terminals 300 may access an artificial intelligence-based cloud platform system 100 (hereinafter, referred to as a “cloud platform system 100”) according to an embodiment of the present disclosure through a communication network 500, and a reading model may be generated using a graphical user interface for designing a learning model and for generating the reading model, the graphical user interface being provided by the cloud platform system 100 according to the embodiment of the present disclosure.

The cloud platform system 100 according to an embodiment of the present disclosure may include a dataset storage unit 110 in which multiple datasets 111 are stored, multiple image processing modules 121, multiple artificial intelligence modules 131, a model learning unit 140, and a medical image reading unit 150 as shown in FIG. 2. Herein, although it is described that the image processing modules 121 may be stored in a first module storage unit 120 and the artificial intelligence modules 131 may be stored in a second module storage unit 130, as an example, the first module storage unit 120 and the second module storage unit 130 do not mean physically separated storage units.

The multiple datasets 111 stored in the dataset storage unit 110 may be classified according to at least one among a body part, a type of modality, a type of disease to be read, and a type of image dimension.

The body part may be a body part to be learned or read, and examples thereof may include the abdomen, brain, head, neck, spine, breasts, chest, gynecological parts, urological parts, heart, vessels, and musculoskeletal system.

Examples of the type of modality may include CT, MRI, MRA, X-Ray, ultrasonography, and PET. Examples of the type of disease to be read may include various diseases or lesions that may be readable using a medical image, for example, a tumor, scoliosis, pneumonia, and diabetic retinopathy. In addition, examples of the type of image dimension may include a 2D image and a 3D image.

Multiple medical images classified according to such a classification criterion may constitute one dataset 111, and the user may perform training using one dataset 111.

The image processing modules 121 may be pre-programmed to perform preprocessing of a medical image and may be modularized. The image processing modules 121 may be divided into a module for preprocessing of a color image, a module for preprocessing of a gray scale image, a module for preprocessing of a binary image, and other modules, and a detailed description thereof will be described later.

In the artificial intelligence modules 131, an artificial intelligence algorithm may be pre-programmed and modularized. In the present disclosure, as an example, a deep learning-based neural network algorithm may be modularized and thus the artificial intelligence modules 131 may be constructed, and a detailed description thereof will be described later.

The model learning unit 140 may include: a learning model design unit 141 supporting the design of a learning model; and a reading model generation unit 142 generating a reading model by training the learning model designed by the learning model design unit 141.

In the present disclosure, for convenience of description, a description is given distinguishing between a “learning model” and a “reading model”. The learning model may mean a model in a process of designing, by the user, a model through the graphical user interface provided by the cloud platform system 100 according to the present disclosure, and may define a state in the design process before actual training is performed. The reading model may mean a model after the designed learning model is trained using the datasets 111, and may define a state in which a parameter, such as a weight value, is updated.

The learning model design unit 141 may provide a user terminal 300 that has had access through a web browser, with the graphical user interface for designing an artificial intelligence-based learning model. In addition, as described above, the reading model generation unit 142 may generate a reading model by training the learning model designed by the learning model design unit 141.

Herein, in designing a learning model, the learning model design unit 141 may provide the graphical user interface for designing a learning model through selection of and drag-and-drop of the datasets 111, of the image processing modules 121, and of the artificial intelligence modules 131, and through line connection between modules. Accordingly, the user is able to easily design a learning model by simple manipulation, such as a drag-and-drop operation, of the datasets 111, the modularized image processing algorithms and artificial intelligence algorithms, and is able to generate a reading model.

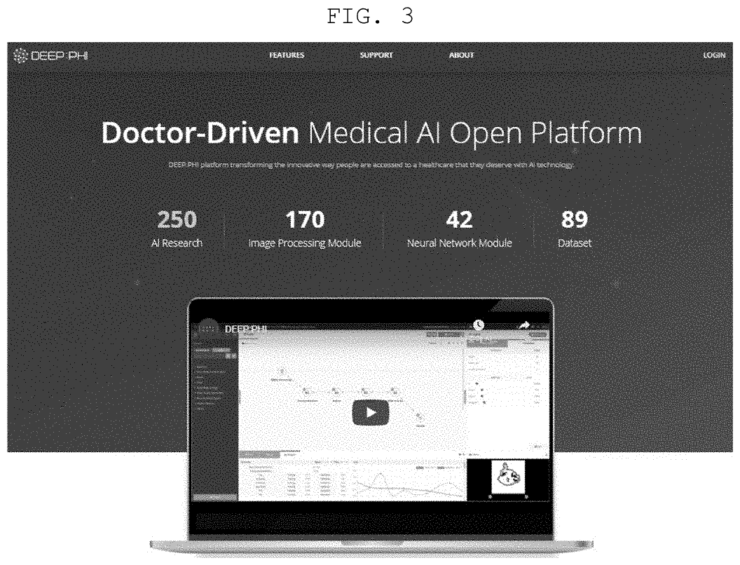

Hereinafter, a process of creating a new project for designing a learning model and for generating a reading model, by using a graphical user interface that a cloud platform system 100 according to an embodiment of the present disclosure provides will be described with reference to FIGS. 3 to ?. Herein, the process of creating a new project may be performed by the learning model design unit 141.

First, when the user uses the user terminal 300 to access a website that the cloud platform system 100 provides, a main screen as shown in FIG. 3 is displayed on the screen of the user terminal 300.

Next, the user logs in with the user's ID and password (the top right in FIG. 3), and when the user clicks on a my page menu, a my page screen is displayed on the screen of the user terminal 300.

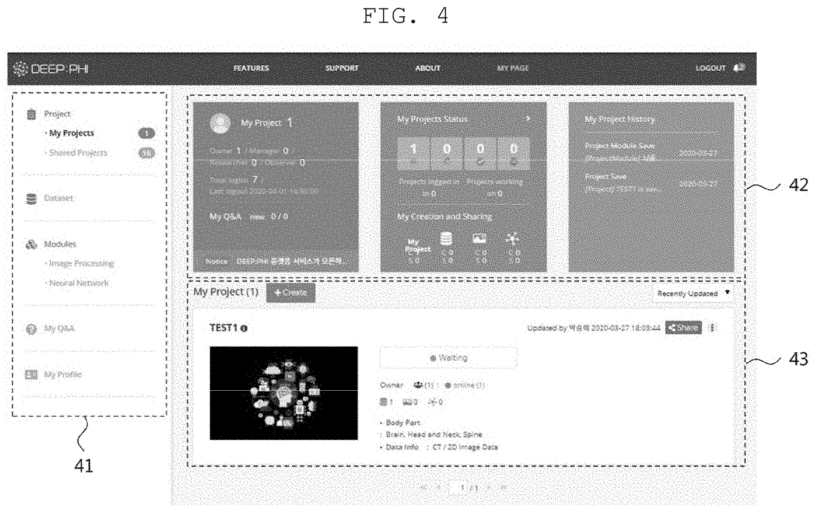

FIG. 4 is a diagram illustrating an example of a my page screen that the cloud platform system 100 according to the present disclosure provides. Referring to FIG. 4, according to an embodiment of the present disclosure, the my page screen may include a my page menu window 41, a project status window 42, and a my project list window 43.

The my page menu window 41 may include a project menu (Project) composed of My Projects and Shared Projects, a dataset menu (Dataset), and a module menu (Modules), and may further include a Q&A menu (My Q&A) and a my profile menu (My Profile).

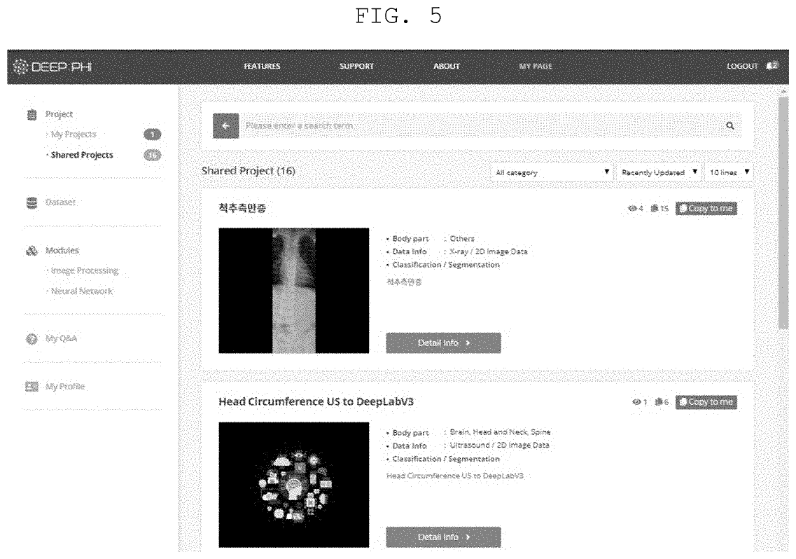

When My Projects is clicked, a screen as shown in FIG. 4 is displayed. When Shared Projects is clicked, lists of projects set to be shared among projects generated by other users are displayed on the screen as shown in FIG. 5.

Similarly, when the dataset menu is clicked, a list of datasets that the cloud platform system 100 currently possesses is displayed on the right side of the my page menu window 41 as shown in FIG. 5. When the dataset is clicked, detailed information is provided so as to be checked. Herein, the datasets that the cloud platform system 100 possesses may be displayed in a dataset list on a modular screen, and a description thereof will be described later.

The module menu may include an image processing item (Image processing) and a neural network item (Neural Network). When the image processing item is clicked, a list of image processing modules 121 that the cloud platform system 100 currently possesses is displayed on the right side of the my page menu window 41 as shown in FIG. 5. Similarly, when the neural network item is clicked, a list of artificial intelligence modules 131 that the cloud platform system 100 possesses is displayed on the right side of the my page menu window 41 as shown in FIG. 5. Herein, the image processing modules 121 and the artificial intelligence modules 131 that the cloud platform system 100 possesses may be displayed in a module list display window 91, which will be described later, on a modular screen, and a description thereof will be described later.

Referring back to FIG. 4, in the project status window 42 on the my project screen, the following may be displayed: project status of a currently logged-in user; resource status; notice for each project; information on the status of all projects currently registered in the cloud platform system 100; and information on project execution history of a current user.

In the my project list window 43, a list of projects executed by a currently logged-in user may be displayed. FIG. 4 shows an example in which one project has been executed.

Herein, when a “+Create” item in the my project list window 43 is clicked, an attribute input window for inputting attribute information of a reading model to be generated as a new project is displayed on the graphical user interface.

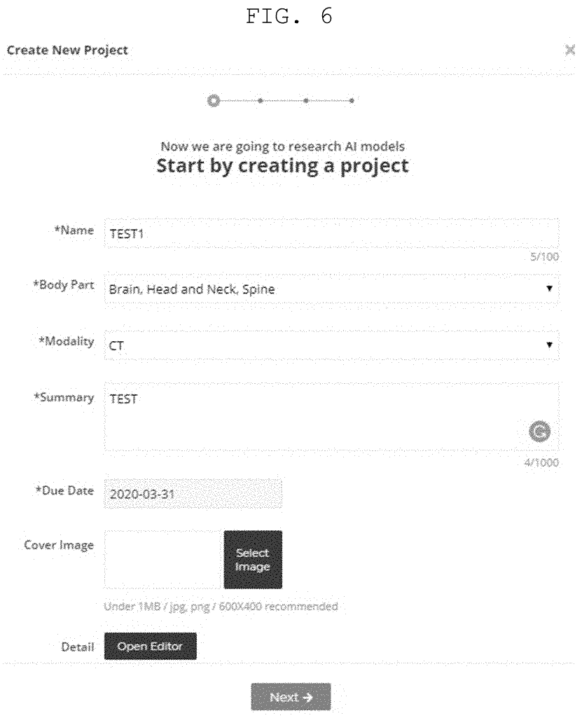

First, when the “+Create” item is clicked to create a new project, a project basic information input pop-up window as shown in FIG. 6 is displayed as an attribute input window on the screen of the user terminal 300.

In the project basic information input pop-up window, the following may be displayed: items for inputting a project name, selecting a body part, selecting a type of modality, inputting a project summary, inputting a project due date, and selecting a cover image. Herein, a body part and a type of modality may be included in attribute information of a reading model to be generated as a new project.

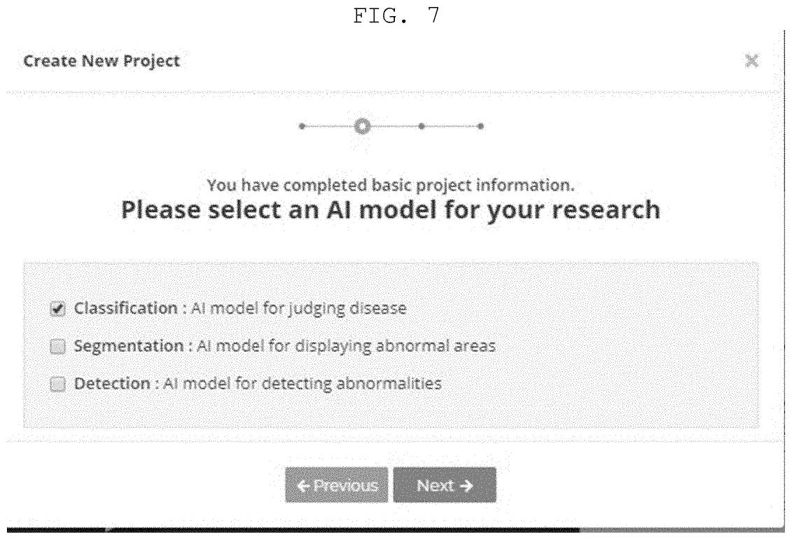

After attribute information is input using the project basic information input pop-up window, when a next button (Next) is clicked, a model type selection pop-up window for selecting a type of artificial intelligence model is displayed as an attribute input window on the graphical user interface screen as shown in FIG. 7.

In the present disclosure, examples of the type of artificial intelligence model include a classification model, an object detection model, and a segmentation model as shown in FIG. 7, but no limitation thereto is imposed. Herein, as an example, one or two or more of the artificial intelligence models in the model type selection pop-up window shown in FIG. 7 may be selected.

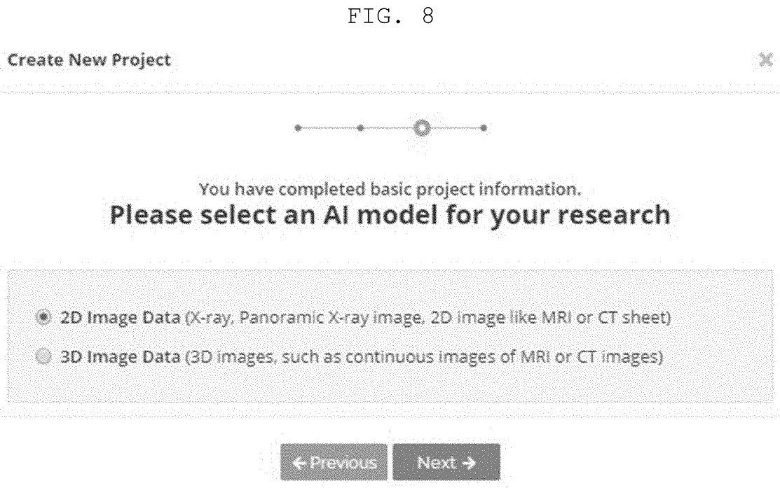

After a type of artificial intelligence model is selected, when a next button (Next) is clicked, a data type selection pop-up window for selecting a type of dataset 111 including 2D and 3D and to be learned is displayed as an attribute input window in the graphical user interface as shown in FIG. 8. Herein, a 2D or 3D image dataset 111 may be selected, or both may be selected.

Through the above-described process, when the type of artificial intelligence model and the 2D or 3D dataset 111 are selected, the type of artificial intelligence model and the type of dataset 111 are registered as the above-described attribute information.

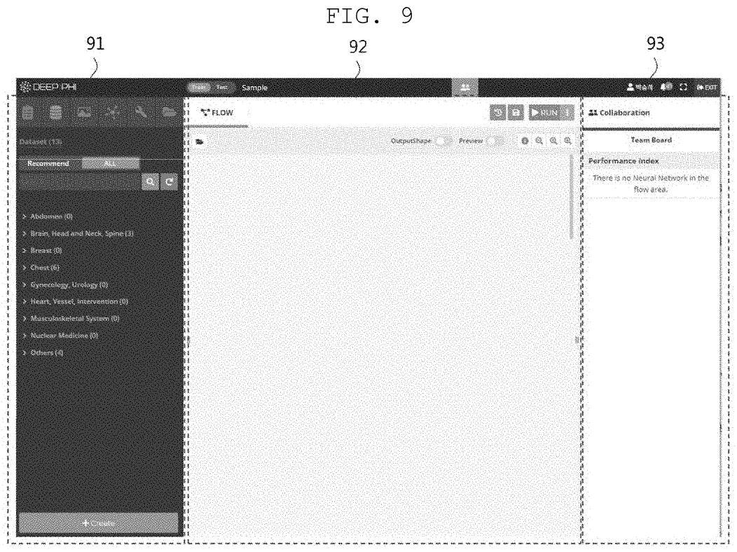

When the process of registering the attribute information is completed, the learning model design unit 141 displays a modular screen on the graphical user interface as shown in FIG. 9.

The modular screen may include the module list display window 91, a learning model design window 92, and an information display window 93.

In the module list display window 91, a list of multiple datasets 111, a list of multiple image processing modules 121, and a list of multiple artificial intelligence models may be displayed.

Herein, in the present disclosure, when the learning model design unit 141 displays lists in the module list display window 91, only the lists of the datasets 111, the image processing modules 121, and the artificial intelligence modules 131 that are matched with the attribute information input through the attribute input window as described above are displayed. Through this, for a user who lacks knowledge in the field of artificial intelligence, only modules matched with attribute information of a reading model that the user wants to design and generate may be displayed on the list, thus making the access easier.

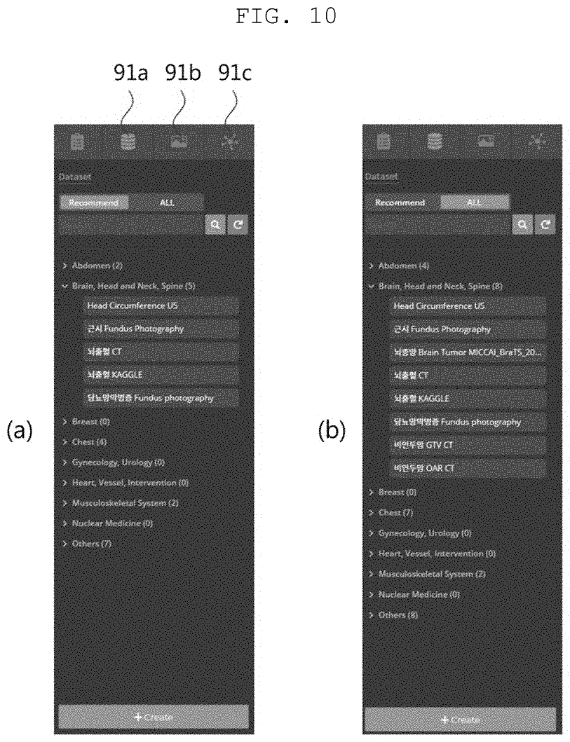

FIG. 10(a) is a diagram illustrating an example of the module list display window 91 in which only the lists matched with the attribute information are displayed. FIG. 10(b) is a diagram illustrating an example of the module list display window 91 in which all lists are displayed. FIG. 10 shows an example of a list of datasets 111.

Referring to FIG. 10, an entire list selection item (ALL) and a recommended list selection item (Recommend) may be provided in the module list display window 91 according to the present disclosure. When the entire list selection item (ALL) is clicked, the entire list of datasets 111 is displayed as shown in FIG. 10(b). When the recommended list selection item (Recommend) is clicked, only a recommended list, specifically, a list of datasets 111 matched with the attribute information registered in advance, is displayed as shown in FIG. 10(a).

In the meantime, in the module list display window 91, a dataset selection item 91a, an image processing selection item 91b, and an artificial intelligence selection item 91c may be provided. When the dataset selection item 91a is clicked, the learning model design unit 141 displays a list of datasets 111 in the module list display window 91 (see FIG. 10).

Similarly, when the image processing selection item 91b is selected and clicked, the learning model design unit 141 displays a list of image processing modules in the module list display window 91. Herein, similarly to the case of the datasets 111, an entire list selection item (ALL) or a recommend list selection item (Recommend) may be selected and thus an entire list or a matched recommended list may be displayed.

When the artificial intelligence selection item 91c is clicked, the learning model design unit 141 displays a list of artificial intelligence modules 131 in the module list display window 91. Similarly, an entire list selection item (ALL) or a recommend list selection item (Recommend) may be selected and thus an entire list or a matched recommended list may be displayed.

In the meantime, the cloud platform system 100 according to an embodiment of the present disclosure may include, as shown in FIG. 2, a model storage unit 160 in which pre-generated reading models and pre-designed learning models are stored.

Herein, the reading models or learning models stored in the model storage unit 160 may be registered by the user of the cloud platform system 100 according to the present disclosure by sharing the learning models designed by the user or reading models generated by the user. Other users may be allowed to access the reading models or learning models through sharing.

Herein, the learning model design unit 141 may search the model storage unit 160 for the reading model or learning model matched with the attribute information input through the attribute input window, and may display, in the graphical user interface, a recommended model list window in which a preset number of matched learning models or reading models in a list are displayed. In the present disclosure, as an example, the recommended model list window is displayed in the form of a pop-up window before switching to a modular screen takes place as the attribute information is input through the attribute input window. When the user clicks any one of the recommended models in the recommended model list window, the clicked learning model or reading model is displayed in the modular screen, and on the basis of this, a new learning model may be designed through modification.

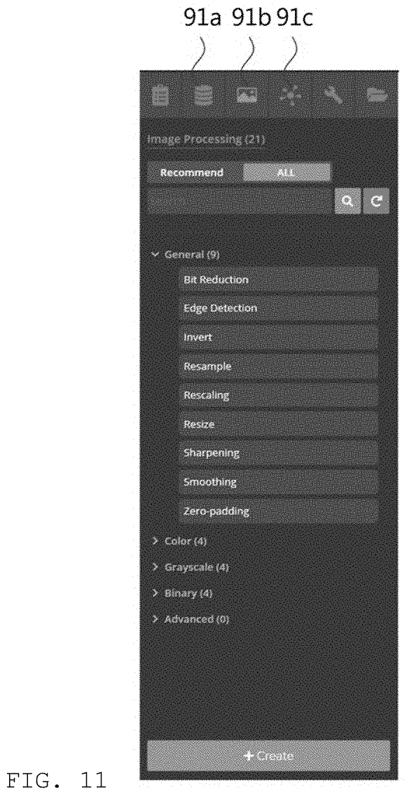

FIG. 11 is a diagram illustrating an example in which a list of image processing modules 121 is displayed in the module list display window 91 in the cloud platform system 100 according to an embodiment of the present disclosure. In the present disclosure, as an example, the list of image processing modules 121 displayed in the module list display window 91 may be displayed, being divided into multiple preprocessing function-based groups.

Herein, the preprocessing function-based groups may include a color preprocessing group (Color), a grayscale preprocessing group (Grayscale), and a binary preprocessing group (Binary). In addition, the preprocessing function-based groups may include a general preprocessing group (General).

In the color preprocessing group, a list of image processing modules 121 operating for an 8-bit or more color image may be displayed.

The image processing modules 121 belonging to the color preprocessing group may include a color mode conversion module, a color-to-grayscale module, a color gamma correction module, and a color histogram equalization module.

The color mode conversion module may be a module for conversion between various color spaces. The color-to-grayscale module may be a module for converting a color image to a grayscale image. The color gamma correction module may be a module for performing gamma correction of a color image. The color histogram equalization module may be a module for equalizing a histogram of a color image.

Grayscale preprocessing modules may include a grayscale gamma correction module, a grayscale histogram equalization module, a morphological conversion module, and a threshold conversion module.

The gamma correction module and the grayscale histogram equalization module may perform gamma correction and histogram equalization of a grayscale image, respectively. The morphological conversion module may be a module for performing morphological conversion of a grayscale image. The threshold conversion module may be a module for performing binarization of a grayscale image to have only two values, black and white.

Binary preprocessing modules may include a contour detection module, a convex hull module, a binary-image morphological conversion module, and a skeletonize module.

The contour detection module may be a module for detecting a contour by connecting pixels having the same pixel value. The convex hull module may be a module for generating the smallest mask containing all white pixel values within an image. The skeletonize module may be a module for making only the skeleton in an image remain.

The general preprocessing group (General) may include a list of image processing modules except the above-described image processing modules 121 applied only to a color image, grayscale image, or binary image.

As in the example shown in FIG. 11, the general preprocessing group (General) may include a bit reduction module (Bit Reduction), an edge detection module (Edge Detection), an inversion module (Invert), a resampling module (Resample), a rescaling module (Rescaling), a resizing module (Resize), a sharpening module (Sharpening), a Smoothing module (Smooding), a zero-padding module (Zero-padding).

The bit reduction module (Bit Reduction) may be a module for converting an image into an 8-bit integer form by reducing a bit value of color used in image expression. The edge detection module (Edge detection) may be a module for finding and binarizing an edge in an image. The inversion module (Invert) may be a module for inverting a pixel value in an image. The resampling module (Resample) may be a module for adjusting the size of an image by using information on a distance between pixels. The rescaling module (Rescaling) may be a module for changing a range of pixel values located in an image sample region, for example, a profile or an entire image. The resizing module (Resize) may be a module for enlarging or reducing the size of an image. The sharpening module (Sharpening) may be a module for applying the effect of emphasizing an edge in an image. The smoothing module (Smoothing) may be a module for applying a blur effect to make an image blurred. The zero-padding module (Zero-padding) may be a module for adding a square shape with a zero value to an original image.

As described above, in designing the learning model, the image processing algorithms required for the preprocessing process of an original medical image may be modularized, and may be grouped according to a preprocessing function to provide a list, so that the user may more easily find an image processing algorithm appropriate for a learning model that the user wants to design.

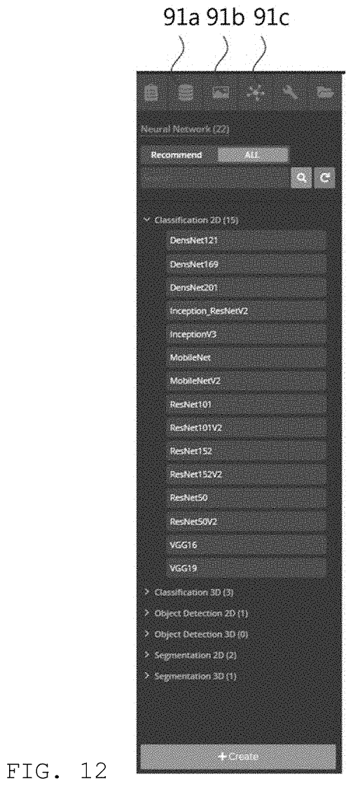

FIG. 12 is a diagram illustrating an example in which a list of artificial intelligence modules 131 is displayed in the module list display window 91 in the cloud platform system 100 according to an embodiment of the present disclosure. In the present disclosure, as an example, the list of artificial intelligence modules 131 displayed in the module list display window 91 may be displayed, being divided into artificial intelligence function-based groups.

As an example, the artificial intelligence function-based groups may include a 2D classification group (Classification 2D), a 3D classification group (Classification 3D), a 2D detection group (Object Detection 2D), a 3D detection group (Object Detection 3D), a 2D segmentation group (Segmentation 2D), and a 3D segmentation group (Segmentation 3D).

In the 2D classification group (Classification 2D), a list of artificial intelligence modules 131 performing classification of a 2D image may be displayed. In the 3D classification group (Classification 3D), a list of artificial intelligence modules 131 performing classification of a 3D image may be displayed. In the 2D detection group (Object Detection 2D), a list of artificial intelligence modules 131 performing object detection of a 2D image may be displayed. In the 3D object detection group (Object Detection 3D), a list of artificial intelligence modules 131 performing object detection of a 3D image may be displayed. In the 2D segmentation group (Segmentation 2D), a list of artificial intelligence modules 131 performing segmentation of a 2D image may be displayed. In the 3D segmentation group (Segmentation 3D), a list of artificial intelligence modules 131 performing segmentation of a 3D image may be displayed.

As the artificial intelligence algorithms performing classification of an image, DensNet, ResNet, and MobileNet algorithms are widely known. As the artificial intelligence algorithms performing object detection, YOLO, SSD, and Retinanet algorithms are known. As the artificial intelligence algorithms performing segmentation, DeepLab, and U-net algorithms are widely known.

Artificial intelligence algorithms designed using the above-described algorithms are modularized and stored as the artificial intelligence modules 131. The user designs a learning model by selecting the artificial intelligence modules 131 displayed in the module list display window 91, whereby more easy design is fulfilled.

Herein, in the modular screen according to an embodiment of the present disclosure, as shown in FIG. 9, the information display window 93 is provided. The learning model design unit 141 may provide various types of information through the information display window 93.

When the user selects any one among the datasets 111, the image processing modules 121, and the artificial intelligence modules 131 displayed in a model list display window, the learning model design unit 141 displays information on the selected dataset 111 or module in the information display window 93.

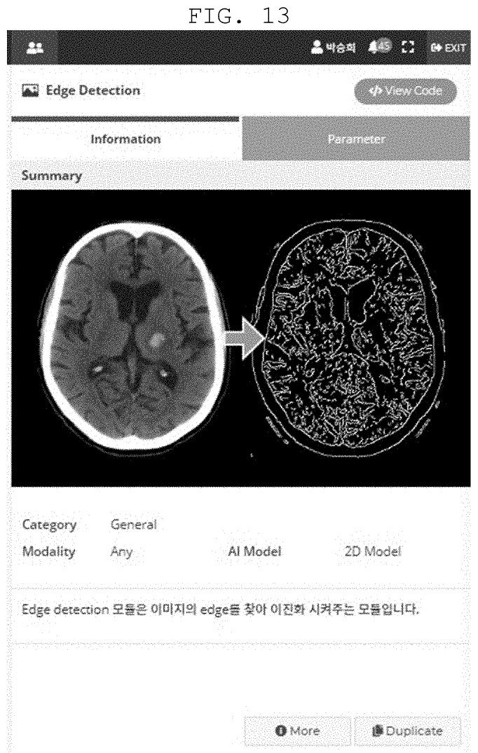

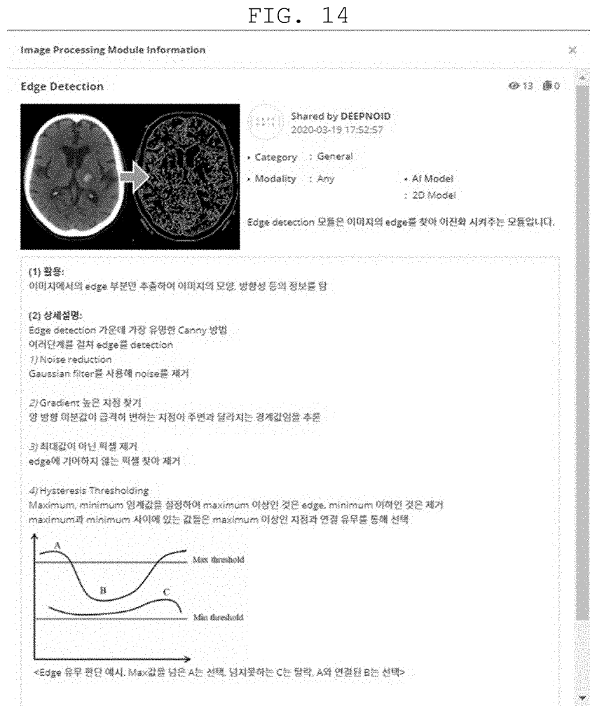

FIG. 13 shows an example of information displayed in the information display window 93 when the edge detection module is selected among the image processing modules 121. As information on the image processing module 121, images before and after processing by the image processing module 121 are displayed as shown in FIG. 13, and simple information on the image processing module 121 is displayed at the bottom, as an example. In addition, to provide more information, when a detailed information button (More) is clicked, more detailed information on the image processing module 121 is provided as shown in FIG. 14.

Similarly, when any one of the artificial intelligence modules 131 is selected, a description of the function, main use, and layer structure of the selected artificial intelligence module 131 is provided, and when a detailed information button (More) is clicked, detailed information is provided.

Hereinafter, a process of designing a learning model through a modular screen will be described.

When the user selects one of the lists displayed in the module list display window 91 and drags and drops the same into the learning model design window 92, a module icon (or an icon for the dataset 111) is generated in the learning model design window 92 in response to the drag and drop.

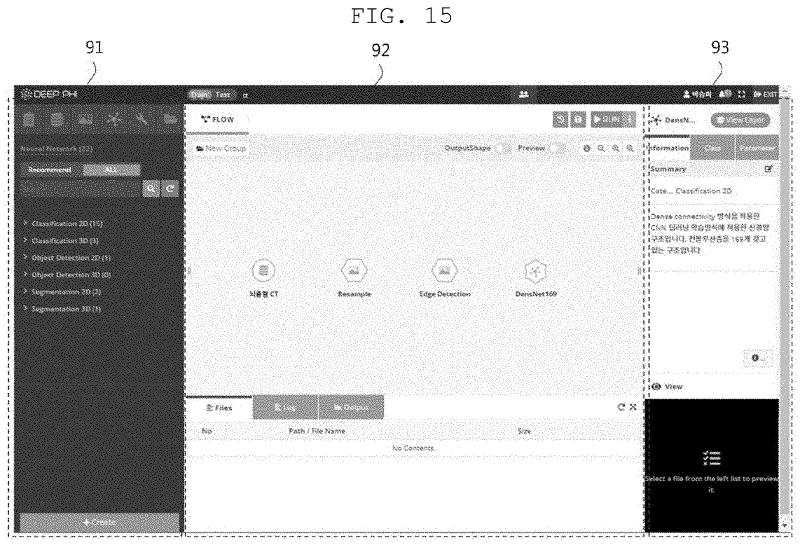

FIG. 15 shows an example of a state in which one dataset 111, two image processing modules 121, and one artificial intelligence module 131 are dragged and dropped into the learning model design window 92 and an icon for the dataset 111 and a module icon are thus displayed.

When the user uses a mouse cursor to connect the icon for the dataset 111 and the module icons in a line, the learning model design unit 141 generates a learning model by using the line connection between the icons as a data flow.

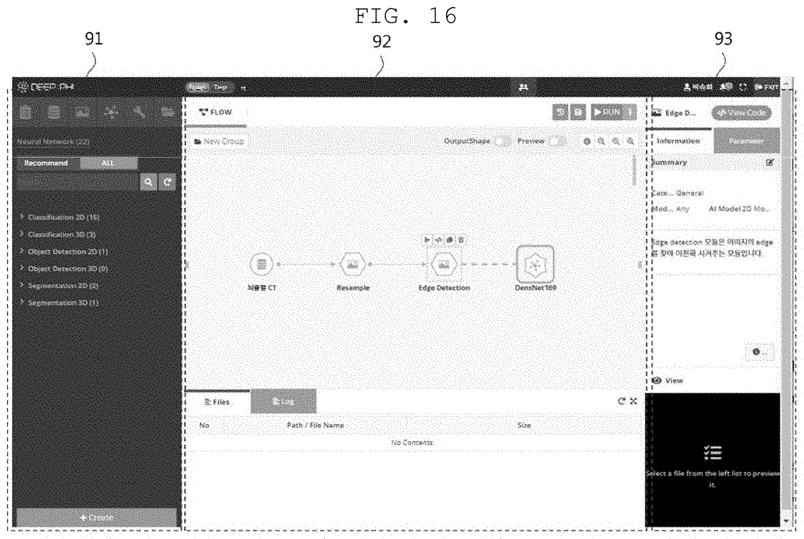

FIG. 16 shows a state in which the icons displayed in the learning model design window 92 are connected in a line therebetween. The learning model design unit 141 displays that line connection is possible when a mouse cursor is positioned on an icon. When the mouse cursor is moved to another icon, the direction in which the cursor moves between the two icons is recognized as a data flow and the line connection in the form of an arrow is displayed.

As described above, it is possible to design a learning model only with simple operations, such as drag and drop, and line connection between icons, thus enabling easy design of a learning model despite lack of knowledge in the field of artificial intelligence.

In the meantime, the cloud platform system 100 according to an embodiment of the present disclosure may further include multiple layer modules as shown in FIG. 2. The layer modules in which layers applied to the configuration of an artificial intelligence algorithm are modularized by function may be stored in the layer storage unit 170.

The artificial intelligence algorithm may be formed in a network structure of multiple layers, and the layers for constructing the artificial intelligence algorithm may include a core layer, a convolution layer, a pooling layer, a merge layer, and a normalization layer.

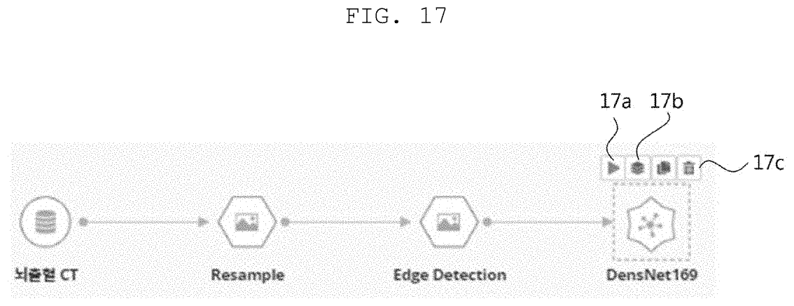

Herein, when an icon for an artificial intelligence model displayed in the learning model design window 92 is selected, the learning model design unit 141 displays buttons for performing functions related to the selected icon as shown in FIG. 17. Reference numeral 17a denotes an individual execution button 17a, which will be described later. Reference numeral 17b denotes a layer entry button 17b for checking and modifying the design structure of the artificial intelligence model. Reference numeral 17c denotes a delete button 17c for deleting the icon.

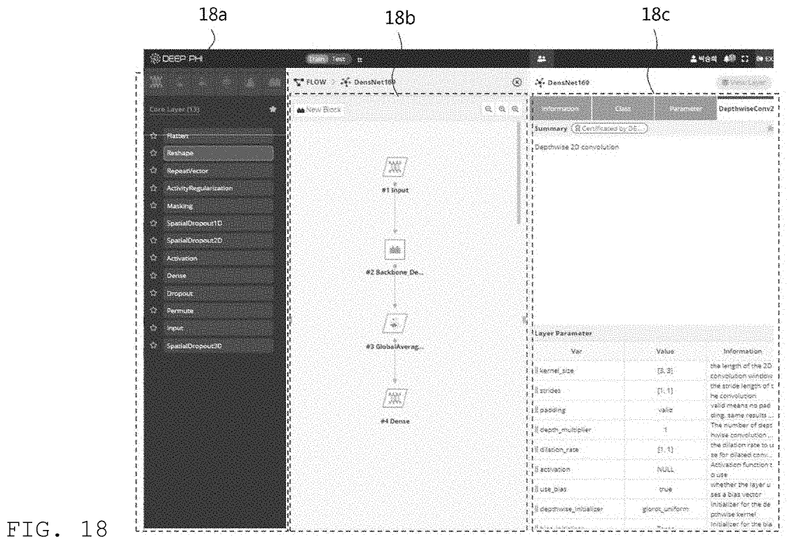

When the layer entry button 17b is clicked, the learning model design unit 141 displays a layer list display window 18a in which a list of layer modules is displayed, and an artificial intelligence design window 18b in the graphical user interface as shown in FIG. 18.

In addition, as in the above-described modular screen, when a layer module or a layer block displayed in the layer list display window 18a is selected, the learning model design unit 141 displays a layer information display window 18c in which information on the layer module or layer block is displayed, in the graphical user interface. Herein, in the layer information display window 18c, parameter information may be displayed. In the present disclosure, the parameter may be modified, as an example.

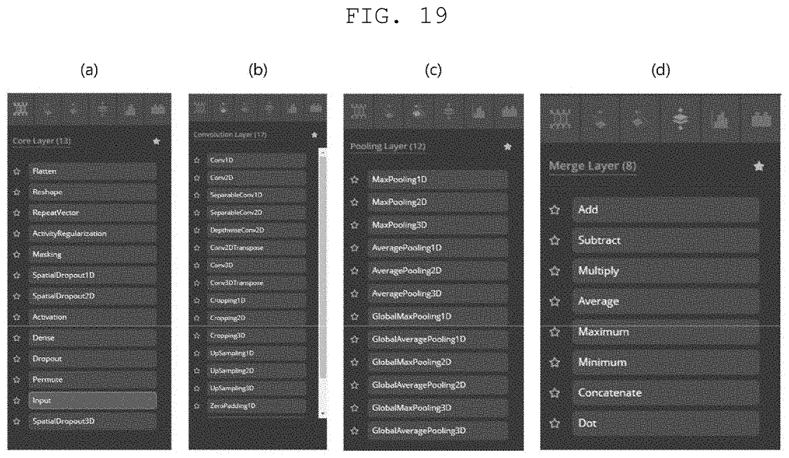

In the layer list display window 18a, multiple lists of layers may be displayed, being divided by group described above. As shown in FIG. 19, when an icon button located on the top of the layer list display window 18a is clicked, lists corresponding to the respective groups are displayed in the layer list display window 18a.

Herein, when the user selects any one of the lists of layer modules and moves the same to the artificial intelligence design window 18b through drag and drop, the learning model design unit 141 displays icons for the selected layer modules in the artificial intelligence design window 18b.

In addition, as in the design of a learning model, when layer icons displayed in the artificial intelligence design window 18b are connected in a line, the learning model design unit 141 generates an artificial intelligence module 131 by using the line connection between the layer icons as a data flow.

The user may use the layer list display window 18a and the artificial intelligence design window 18b to generate the artificial intelligence module 131 designed by the user, and the artificial intelligence module may be shared so that other users use the artificial intelligence module. In addition, when the user designs another learning model, the user reuses the previously generated artificial intelligence module 131 or use artificial intelligence modules 131 designed by others. That is, generation, sharing, and reuse of various artificial intelligence modules 131 are possible.

In addition, the cloud platform system 100 according to the present disclosure may further include multiple layer blocks that are composed of at least two or more layers and modularized for generating an artificial intelligence module 131.

Herein, the learning model design unit 141 may also display a list of layer blocks in the layer list display window 18a, and may support generation of a layer block through drag and drop of a list of layer modules displayed in the layer list display window 18a into the artificial intelligence design window 18b, and through line connection.

In addition, a layer block may be generated through drag and drop of multiple layer modules and multiple layer blocks, and line connection. That is, the following structure may be provided: the design of a network structure of layer modules enables modularization of layer blocks, the design of a network structure of layer modules and layer blocks enables the design of layer blocks, and the design of a network structure of layer modules and layer blocks enables the design and manufacture of an artificial intelligence mode.

Such a design structure makes it possible to visually and simply check the design structure of a final artificial intelligence module 131, to determine the structure in the form of finding a detailed structure, and to easily understand a network structure, specifically, layer modules, layer blocks, and a line connection structure.

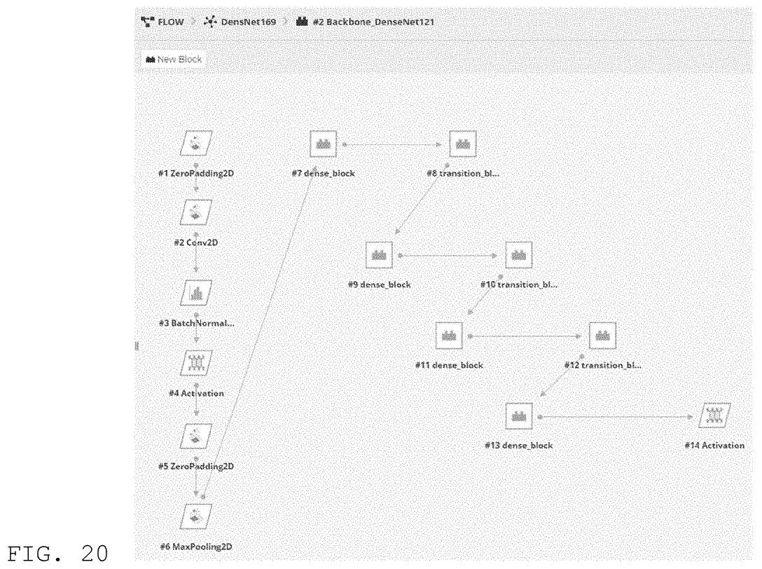

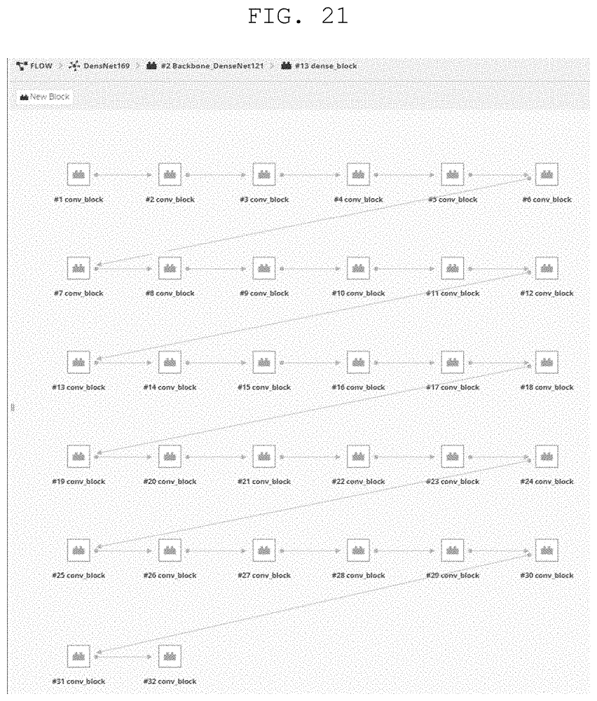

That is, a detailed network structure of an artificial intelligence model in the network structure of the learning model shown in FIG. 17 is as shown in FIG. 18. In addition, layer block “#2 Blackbone_DenseNet121” shown in FIG. 18 has the detailed network structure as shown in FIG. 20, and layer block “#12 dense_block” shown in FIG. 20 has the detailed network structure as shown in FIG. 21.

Herein, when the user sets storing or sharing of the artificial intelligence modules 131 or layer blocks generated through the artificial intelligence design window 18b, the learning model design unit 141 updates a list of the artificial intelligence modules 131 or layer blocks in the layer list display window 18a so that other users are able to use the same.

That is, the user may design various types of layer blocks by using the layer modules in which basic layer algorithms constituting an artificial intelligence algorithm are modularized, and may design other layer blocks through the design of the network of layer modules and layer blocks, thereby generating a final artificial intelligence module 131.

In addition, as described above, through sharing of layer blocks or artificial intelligence modules 131 between users, a user may redesign using the layer blocks or artificial intelligence modules 131 designed by others and may reuse the artificial intelligence modules 131 or layer blocks designed by the user, thus providing an environment that may be easily used for the design of a new learning model.

Referring back to FIG. 6, after the learning model is designed using the learning model design window 92 through the above-described process, when a “RUN” button provided in the learning model design window 92 is clicked, the reading model generation unit 142 trains the learning model designed by the learning model design unit 141 and generates a reading model.

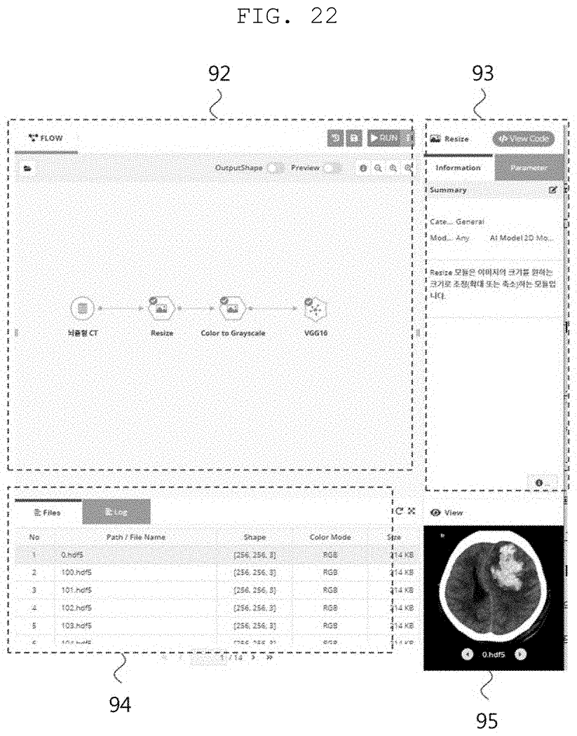

When normal training is performed by the reading model generation unit 142, an icon displayed in the learning model design window 92 is changed such that completion of normal operation is visually checked as shown in FIG. 22. In the present disclosure, the visualization is provided in such a manner that change from gray to green takes place and a check mark appears on the icon, as an example.

Conversely, when an error occurs in training, the error is visually displayed in such a manner that the icon for the image processing module 121 or artificial intelligence module 131 in which the error has occurred is displayed in red with an error message.

Herein, the reading model generation unit 142 according to the present disclosure may be provided to operate in either an entire learning execution mode or an individual module execution mode. The entire learning execution mode may be a process of training, on the basis of the line connection and the icons displayed in the learning model design unit 141, the whole sequentially by using the line connection as a data flow through the click of the “RUN” button as described above.

On the other hand, the individual module execution mode may be operated by clicking the individual execution button 17a that appears when the cursor is placed on the icons for the image processing module 121 and the artificial intelligence module 131 as shown in FIG. 17.

Herein, with the cursor positioned on a particular icon, when the individual module execution mode is performed by clicking the individual execution button 17a activated for the icon, the reading model generation unit 142 executes only the image processing module 121 or the artificial intelligence module 131 corresponding to the icon for which the individual execution button 17a is selected, according to the data flow based on the line connection of the dataset 111 corresponding to a data icon.

For example, when the individual execution button 17a for icon “Color to Grayscale” in the learning model design window 92 of FIG. 22 is clicked, the reading model generation unit 142 executes only the image processing modules 121 corresponding to “Resize” and “Color to Grayscale” sequentially by using the dataset 111 corresponding to cerebral hemorrhage CT.

Through this, in the process of designing the learning model, it is possible to check whether normal operation takes place, for example, by executing only “Resize” during preprocessing, without performing training after the entire design is completed. Therefore, by checking errors occurring in the design process in advance, the time required for the design process may be significantly reduced.

In addition, when operating in the entire learning execution mode and the individual module execution mode, the reading model generation unit 142 stores a processing result on the basis of the image processing module 121 and the artificial intelligence module 131.

In addition, after either the image processing module 121 or the artificial intelligence module 131 or both are changed through the learning model design window 92, when either the entire learning execution mode or the individual mode execution mode is performed, the reading model generation unit 142 fetches and applies the previous processing result up to the unchanged line connection in the data flow according to the line connection, thereby saving the time required for re-executing the image processing module 121 or the artificial intelligence module 131 that has already been executed.

Referring to FIG. 22, after the user executes “Resize” in the individual module execution mode, when “Color to Grayscale” and “VGG16” are designed and “Color to Grayscale” is executed in the individual module execution mode, or execution in the entire learning execution mode takes place through the click of the “RUN” button, the previous processing result is fetched and applied for “Resize” that has already been executed and has a result stored and only “Color to Grayscale” and/or “VGG16” are executed.

In addition, as shown in FIG. 22, after execution in the entire learning execution mode is completed, when “Color to Grayscale” is deleted and replaced with another image processing module 121 and the entire learning execution mode or the individual module execution mode is performed, the previous processing result is fetched and applied for “Resize”, similarly. Herein, even though the module after the changed module according to the data flow is not modified, it is proper that the module is executed again, because the processing result of the previous step is changed.

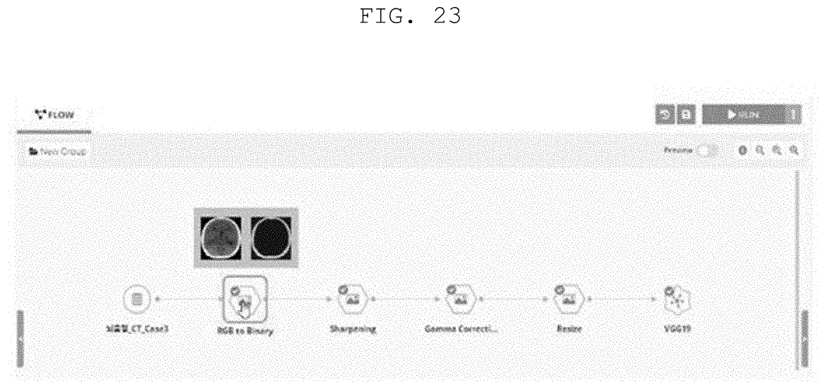

In the meantime, when a module icon corresponding to the image processing module 121 displayed in the learning model design window 92 is selected, the learning model design unit 141 displays images before and after image processing of the image processing module 121 in a region near the module icon, for example, above the module icon as shown in FIG. 23.

Through this, with only a simple operation of the mouse, in the current preprocessing process of the learning model designed by the user, the user is able to easily check images before and after images processed by the respective image processing modules 121.

Herein, with images before and after image processing displayed near the module icon, when the scroll of the mouse is recognized, the learning model design unit 141 changes the images included in the dataset 111 to display the images before and after image processing.

In addition, in the case in which the module icon corresponding to the image processing module 121 displayed in the learning model design window 92 is selected, when a data list display window 94 in which lists of medical images constituting the datasets 111 are displayed and any one of the lists displayed in the data list display window 94 are selected, the learning model design unit 141 displays the medical images in the list, that is, an image display window 95 in which the images are displayed, in the graphical user interface.

In addition, the learning model design unit 141 may display the medical images displayed in the image display window 95 and images before and after preprocessing of the image processing module 121. Through this, the user may manually select the medical images constituting the dataset 111 one by one, and may visually check a result of preprocessing.

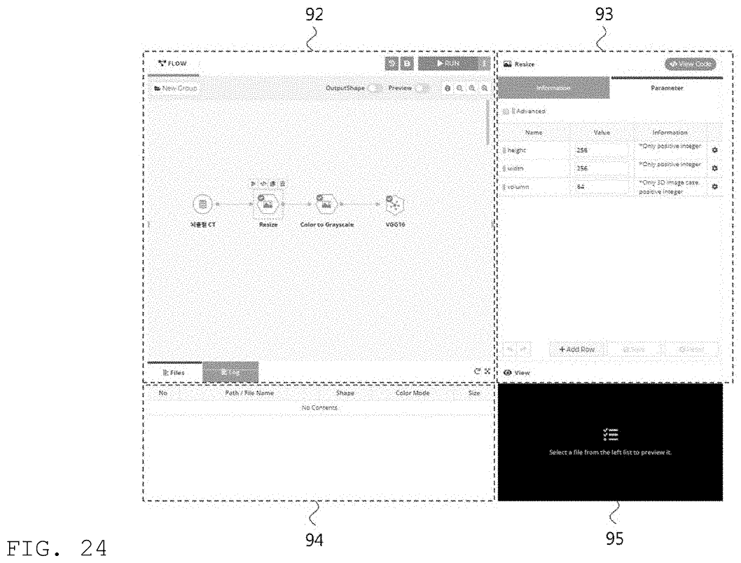

In the meantime, when any one of the icons corresponding to the image processing modules 121 displayed in the learning model design window 92 is selected, the learning model design unit 141 according to the present disclosure displays an information display window 93 in which detailed information on the selected image processing module 121 is displayed, in the graphical user interface. FIG. 24 shows an example of the information display window 93 when the image processing module 121 is selected, and FIG. 9 shows an example of an initial information display window 93 in the modular screen.

In addition, detailed information (Information) and parameter information (Parameter) are displayed together in the information display window 93, and the parameter information determines the preprocessing process of the processing module.

For example, parameters of edge detection may include the maximum threshold (Threshold_max) and the minimum threshold (Threshold_min). Parameters of resampling may include a spacing value. Parameters of gamma correction may include a gamma value. Parameters of histogram equalization may include a kernel size and a limit value.

Herein, the learning model design unit 141 may be set such that values of the parameter information displayed in the information display window 93 may be changed. Therefore, even though the user uses the image processing modules 121 modularized, the user may generate his/her own image processing module 121 by changing the parameter values of the image processing modules 121. Herein, when the reading model generation unit 142 executes the generated image processing module 121, the changed parameters are applied for execution.

Through this, as described above, a result of image processing may be immediately checked by changing the parameters in the individual module execution mode, so that a learning model more may be designed efficiently in terms of time.

As described above, when design of a learning model and generation of a reading model through training are completed, a training result, specifically, a final artificial intelligence model to which a parameter, such as a weight value, is applied is generated. The medical image reading unit 150 reads a medical image to be read, by using the finally generated artificial intelligence model.

Although several embodiments of the present disclosure have been illustrated and described, it will be understood by those skilled in the art that various modifications to the embodiments may be made without departing from the scope or spirit of the present disclosure. The scope of the present disclosure is defined by the appended claims and their equivalents.

### DESCRIPTION OF THE REFERENCE NUMERALS IN THE DRAWINGS

100: cloud platform system
111: dataset 121: image processing module
131: artificial intelligence module 141: learning model design unit
142: reading model generation unit 150: medical image reading unit
160: model storage unit 170: layer storage unit

### INDUSTRIAL APPLICABILITY

The present disclosure may be applied to an artificial intelligence-based cloud platform for reading a medical image.

1. A cloud platform system for reading a medical image, the cloud platform system comprising:
multiple image processing modules pre-programmed to perform preprocessing of the medical image and modularized;
multiple artificial intelligence modules in which an artificial intelligence algorithm is pre-programmed and modularized;
multiple layer modules in which layers applied to a configuration of the artificial intelligence algorithm are modularized by function;
a learning model design unit providing a graphical user interface for designing an artificial intelligence-based learning model to a user terminal that has had access through a web browser; and
a reading model generation unit generating a reading model by training the learning model designed by the learning model design unit,
wherein the learning model design unit configured to
display a layer list display window in which a list of the multiple layer modules is displayed and an artificial intelligence design window in the graphical user interface,
display, when the list displayed in the layer list display window is dragged and dropped into the artificial intelligence design window, layer icons of the layer modules in the artificial intelligence design window, and
generate the artificial intelligence module by using line connection between the layer icons as a data flow when the layer icons displayed in the artificial intelligence design window are connected in a line.

2. The cloud platform system of claim 1, further comprising:
multiple layer blocks composed of at least two or more layers and modularized for generating the artificial intelligence module,
wherein the learning model design unit is configured to
display a list of the multiple layer blocks in the layer list display window, and
support generation of the layer block through drag and drop of the list of the layer modules displayed in the layer list display window into the artificial intelligence design window, and through line connection.

3. The cloud platform system of claim 2, wherein the learning model design unit is configured to
display a module list display window in which a list of the multiple image processing modules and a list of the multiple artificial intelligence modules are displayed and a learning model design window in the graphical user interface,
display, when at least one of the lists displayed in the module list display window is dragged and dropped into the learning model design window, module icons corresponding to the image processing modules or the artificial intelligence modules in the learning model design window, and
generate the learning model using line connection between the module icons as a data flow when the module icons are connected in a line.

4. The cloud platform system of claim 3, wherein the learning model design unit is configured to
display a view button when the module icon corresponding to the artificial intelligence module is selected, and
display, when the view button is selected, the layer list display window and the artificial intelligence design window in the graphical user interface, displaying line connection to the layer modules and/or the layer blocks constituting the artificial intelligence module in the artificial intelligence design window.

5. The cloud platform system of claim 3, further comprising:
a dataset storage unit in which multiple datasets classified according to at least one among a body part, a type of modality, a type of disease to be read, and a type of image dimension are stored,
wherein the learning model design unit is configured to
display a list of the multiple datasets in the module list display window,
display, when the dataset displayed in the module list display window is dragged and dropped into the learning model design window, a data icon in the artificial intelligence design window in response to drag and drop, and
generate, when at least one of the module icons is connected to the data icon in a line, the learning model by using line connection to the module icon as a data flow, and
the reading model generation unit is configured to
generate the reading model by training the learning model generated by the learning model design unit, with the dataset corresponding to the data icon.

6. The cloud platform system of claim 3, wherein the list of the multiple image processing modules displayed in the module list display window is displayed, being divided into multiple preprocessing function-based groups, and the preprocessing function-based groups include at least two of the following:
a color preprocessing group in which a list of the image processing modules operating for an 8-bit or more color image is displayed,
a grayscale preprocessing group in which a list of the image processing modules operating for an 8-bit black-and-white image is displayed, and
a binary preprocessing group in which a list of the image processing modules operating for a 1-bit black-and-white image is displayed.

7. The cloud platform system of claim 3, wherein the multiple artificial intelligence modules displayed in the module list display window are displayed, being divided into multiple artificial intelligence function-based groups, and
the artificial intelligence function-based groups include:
a 2D classification group in which a list of the artificial intelligence modules performing classification of a 2D image is displayed,
a 3D classification group in which a list of the artificial intelligence modules performing classification of a 3D image is displayed,
a 2D detection group in which a list of the artificial intelligence modules performing object detection of a 2D image is displayed,
a 3D detection group in which a list of the artificial intelligence modules performing object detection of a 3D image is displayed,
a 2D segmentation group in which a list of the artificial intelligence modules performing segmentation of a 2D image is displayed, and
a 3D segmentation group in which a list of the artificial intelligence modules performing segmentation of a 3D image is displayed.
